(19) United States
(12) Patent Application Publication
(10) Pub. No.: US 2014/0205551 A1
(43) Pub. Date: Jul. 24, 2014

(54) SUNSCREEN

(71) Applicant: Christine Mendrok-Edinger, Basel (CH)

(72) Inventor: Christine Mendrok-Edinger, Basel (CH)

(73) Assignee: DSM IP ASSETS B.V., Heerlen (NL)

(21) Appl. No.: 14/124,132

(22) PCT Filed: Jun. 6, 2012
(86) PCT No.: PCT/EP2012/060656

§ 371 Date: Mar. 28, 2014

(30) Foreign Application Priority Data
Jun. 8, 2011 (EP) 11169194.5
Jun. 15, 2011 (EP) 11169915.3
Jul. 12, 2011 (EP) 11173601.3

(51) Int. Cl. A61K 8/49; A61K 8/67; A61Q 17/00

(52) U.S. Cl. CPC A61K 8/496; A61Q 17/00; A61K 8/678
USPC 424/59; 549/408; 548/260

(57) ABSTRACT

The present invention relates to a composition for topical application comprising at least a benzotriazol derivative and vitamin E or a derivative thereof. Furthermore, the invention relates to compositions that prevent staining of clothes and exhibit a reduced sand adherence on skin.

The present invention relates to a composition for topical application comprising at least a benzotriazol derivative and vitamin E or a derivative thereof. Furthermore, the invention relates to compositions that prevent staining of clothes and exhibit a reduced sand adherence on skin.

Sun care products have evolved considerably over the years. Earlier formulations were intended to protect the user from UV-B radiation as was once thought that UV-B rays were the most important contributors to wrinkling, skin disease, and skin cancer. However, more recent studies have shown that UV-A radiation is equally or even more important in the development of solar damage and skin diseases, such as lupus erythematosus and melanoma and non-melanoma skin cancer. Thus, today's focus is towards eliminating as much of UVA (320-400 nm) and/or UVB (280-320 nm) light as possible. Consequently, there's a constantly increasing need for sun care products exhibiting high SPF's (Sun Protection Factor) and high UVA protection while being photostable.

Benzotriazol derivates such as e.g. benzotriazolyl dodecyl p-cresol have excellent UV-light absorbing properties and could thus contribute significantly to the SPF of a sun care product. However, they have the problem that incorporated into sun care products they tend to stain clothes which is highly undesirable. In addition, the yellow to brownish stains are not readily removable via conventional laundry. Thus, benzotriazol derivates are so far not added to sun care products at high blend ratios. Furthermore, sun care products are often sticky which e.g. leads to an increased sand affinity to the skin which is highly unwanted.

It was therefore the object of the present invention to remedy the disadvantages of the prior art and to develop sun care products comprising specific benzotriazol derivatives which exhibit a reduced cloth staining tendency. Furthermore, such compositions should favourably also exhibit a reduced stickiness resulting in a reduced affinity of sand to the skin.

Surprisingly, it has been found that the addition of Vitamin E or a derivative thereof such as e.g. tocopheryl acetate to topical compositions comprising specific benzotriazol derivatives reduces the staining tendency of these benzotriazol derivatives on clothing. Therefore, the benzotriazol derivatives can be added to topical compositions at elevated blend ratios allowing the formulation of high SPF sun care products.

Furthermore, such compositions exhibited a reduced stickiness.

Thus, the invention relates in one aspect to a topical composition comprising vitamin E or a derivative thereof and at least one benzotriazol derivative of formula (I)

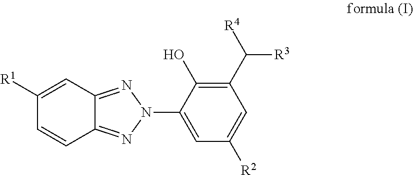

wherein
R¹ is hydrogen; C₁₋₅alkyl; C₁₋₅alkoxy or halogen; preferably hydrogen or chloride; most preferably hydrogen;
R² is hydrogen; C₁₋₂₀alkyl; C₁₋₅alkoxy; C₁₋₅alkoxycarbonyl; C₅₋₁₀cycloalkyl; C₆₋₁₀aryl or aralkyl; preferably hydrogen or C₁₋₅alkyl; most preferably methyl;
R³ is C₁₋₂₀alkyl, C₅₋₁₀cycloalkyl, C₁₋₂₀alkoxy or C₅₋₁₀cycloalkoxy, preferably C₅₋₁₅alkyl or C₅₋₁₅alkoxy; and
R⁴ is hydrogen or C₁₋₅alkyl; preferably hydrogen
characterized in that the benzotriazol derivative of formula (I) is present in an amount ranging from 1 to 20 wt.-%, based on the total weight of the composition.

In another aspect the invention relates to the use of Vitamin E or a derivative thereof for reducing cloth staining caused by a benzotriazol derivative of formula (I).

In yet a further embodiment the invention relates to a method for reducing stains on clothes caused by the use of topical compositions comprising a benzotriazol derivative of formula (I), said method comprising the addition of Vitamin E or a derivative thereof into said topical composition and observing or appreciating the result. It is understood, that the stains are caused by the benzotriazol derivative of formula (I).

The term “C_{x-y}alkyl” as used herein refers to straight-chain or branched alkyl radicals having x to y carbon atoms such as e.g. methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methyl pentyl, 3-methylpentyl, 4-methylpentyl, 1,1-dimethylpropyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,2,2-trimethylpropyl, 1-ethyl-1-methylpropyl, 1-ethyl-2-methylpropyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl or n-eicosyl without being limited thereto.

The term C₅₋₁₀cycloalkyl denotes to unsubstituted or C₁₋₁₀alkyl (mono- or poly-)substituted, in particular C₁₋₅alkyl (mono- or poly-)substituted cyclic, bicyclic or tricyclic hydrocarbon residues such as in particular cyclopentyl, cyclohexyl, cycloheptyl or decahydronaphtyl. Preferably, C₅₋₁₀cycloalkyl denotes to unsubstituted or C₁₋₂alkyl (mono- or poly-)substituted cyclopentyl, cyclohexyl or cycloheptyl such as in particular to unsubstituted or (mono- or poly-)methyl substituted cyclohexyl such as most in particular cyclohexyl or 3,3,5-trimethyl-cyclohexyl.

The term “C_{x-y}alkoxy” as used herein denotes to linear or branched alkoxy-, respectively unsubstituted or (mono- or poly-)substituted cycloalkoxy radicals having from x to y carbon atoms such as e.g. methoxy, ethoxy, propoxy, butyloxy or pentyloxy, 2,5,5-trimethylhexyloxy, 3,5,5-trimethylhexyloxy, isoamyloxy, 2-ethylhexyloxy or 3,3,5-trimethyl-cyclohexyloxy.

The term C₆₋₁₀aryl refers e.g. to naphthyl or phenyl radicals, preferably phenyl.

The term vitamin E or a derivative thereof as used herein refers to tocopherol e.g. available as d-α-tocopherol or dl-α-tocopherol at DSM Nutritional Products Ltd Kaiseraugst, mixed tocopherol (i.e. mixtures of α-, β-, γ-, and/or δ-tocopherol) e.g. commercially available as Mixed Tocopherols 95 at DSM Nutritional Products Ltd, natural tocopherol (R, R, R-α-tocopherol) as well as derivatives thereof such as in particular esters of tocopherol with carboxylic acids such as for example acetic, linoleic, oleic, palmitic and succinic acid. Particular suitable are tocopheryl acetate or tocopheryl linoleate. Tocopheryl acetate is e.g. commercially available as dl-α-Tocopheryl Acetate respectively Tocopheryl Acetate Technical Grade at DSM Nutritional Products Ltd Kaiseraugst.

The term cloth as used herein refers to any textile product made of materials such as cotton, wool, silk, linen, nylon, elastan, satin, polyacryl, fleece, hanf, polyester, viscose, leather as well as mixtures thereof. Particularly affected are white or lightly coloured textiles such as white T-shirts, blouses, skirts, pants, bathing clothes etc.

The amount of the vitamin E or a derivative thereof in the topical compositions according to the present invention is not critical and advantageously selected in the range of 0.05 to 25 wt.-% such as in particular in the range of 0.25 to 10 wt.-% such as most in particular in the range of 0.6 to 5 wt.-% such as in the range of 1 to 5 wt.-% or even in the range of 1 to 3 wt.-%, based on the total weight of the composition.

A particular suitable Vitamin E derivative according to the present invention is tocopheryl acetate. The amount of tocopheryl acetate in the topical compositions according to the present invention is preferably selected in the range of 0.6 to 10 wt.-%, such as most in particular in the range of 1 to 5 wt.-% or even 1.5 to 3 wt.-%, based on the total weight of the composition.

Another particular suitable Vitamin E derivative according to the present invention is tocopheryl linolate. The amount of tocopheryl linolate in the topical compositions according to the present invention is preferably selected in the range of 0.05 to 25 wt.-% such as in particular in the range of 0.6 to 10 wt.-%, such as most in particular in the range of 1 to 5 wt.-% or even 1 to 3 wt.-%, based on the total weight of the composition.

The amount of the at least one benzotriazol derivative of formula (I) in the compositions according to the invention is preferable selected in the range of 2 to 20 wt.-%, such as in the range of 2 to 15 wt.-%, in particular in the range of 4 to 10 wt.-%, and most particular in the range of 4 to 8 wt.-%, based on the total weight of the composition.

In a particular embodiment of the present invention the benzotriazol derivative is selected from compounds of formula (I) wherein R¹ and R⁴ are hydrogen, R² is methyl and R³ is C₆₋₁₀alkoxy such as preferably C₆₋₁₀alkoxy, or C₆cycloalkoxy such as in particular 2,5,5-trimethylhexyloxy, 3,5,5-trimethylhexyloxy, isoamyloxy, 2-ethylhexyloxy or 3,3,5-trimethyl-cyclohexyloxy. Such compounds and their preparation are e.g. disclosed in EP Application No.: 10150832.3. Particularly preferred according to the present invention is a compound of formula (I), wherein R¹ and R⁴ are hydrogen, R² is methyl and R³ is 2-ethylhexyloxy (i.e. 2-(2H-Benzotriazol-2-yl)-6-(2-ethylhexyloxymethyl)-4-methyl-phenol).

In another particular embodiment of the present invention the compound of formula (I) is a compound wherein R¹ and R⁴ are hydrogen, R² is methyl and R³ is undecyl (C₁₁H₂₃) which is commercially available as Tinogard TL [INCI Name: benzotriazolyl dodecyl p-cresol; IUPAC Name: 2-(2H-benzotriazol-2-yl)-6-dodecyl-4-methyl-phenol] at BASF SE Ludwigshafen.

In a further particular embodiment the benzotriazol derivative of formula (I) is benzotriazolyl dodecyl p-cresol and the Vitamin E or derivative thereof is tocopheryl acetate. The benzotriazolyl dodecyl p-cresol is then preferably used in an amount selected in the range of 2 to 20 wt.-% and the tocopheryl acetate in an amount selected in the range of 0.6 to 10 wt.-%. Even more in particular the benzotriazolyl dodecyl p-cresol is used in the range of 2 to 10 wt.-% and the tocopheryl acetate in the range of 0.6 to 5 wt.-% such as in an amount of 1 to 3 wt.-%, based on the total weight of the composition.

In another particular embodiment the benzotriazol derivative of formula (I) is selected from compounds of formula (I) wherein R¹ and R⁴ are hydrogen, R² is methyl and R³ is C₆₋₁₀alkoxy such as in particular C₆₋₁₀alkoxy, or C₆cycloalkoxy such as most in particular 2,5,5-trimethylhexyloxy, 3,5,5-trimethylhexyloxy, isoamyloxy, 2-ethylhexyloxy or 3,3,5-trimethyl-cyclohexyloxy and the Vitamin E or derivative thereof is tocopheryl acetate. Most preferably, the benzotriazol derivative is 2-(2H-Benzotriazol-2-yl)-6-(2-ethylhexyloxymethyl)-4-methyl-phenol. The benzotriazol derivative is then preferably used in an amount selected in the range of 2 to 20 wt.-% and the tocopheryl acetate in an amount selected in the range of 1.5 to 10 wt.-%, based on the total weight of the composition. Even more in particular the benzotriazol derivative is used in the range of 2 to 10 wt.-% and the tocopheryl acetate in the range of 1.5 to 5 wt.-% such as in an amount of 1.5 to 3 wt.-%, based on the total weight of the composition.

In another particular embodiment the compositions according to the present invention are substantially free of a polyglycerol based UV-filter such as e.g. disclosed in [EP Application No's] EP09178503.0, EP09178501.4, EP09178502.2 EP09178495.9, EP09178506.3, EP09178505.5 or EP10150832.3 which are obtainable by a process comprising the steps of ring-opening polymerization of x mol equivalents of glycidol using 1 mol equivalent of a polyol starter unit with y mol equivalents hydroxyl-groups, followed by block copolymerization with z X (x+y) mole equivalents of propylene oxide to form a hyperbranched polyether-polyol backbone carrying (x+y) mol equivalents hydroxyl-groups followed by partial or total esterification, respectively partial or total etherification of the hydroxyl groups with a UV-light absorbing chromophore such as particularly with p-dimethylamino benzoic acid, 3-[1-(4-Hydroxymethyl-phenyl)-meth-(E)-ylidene]-1,7,7-trimethyl-bicyclo[2.2.1]heptan-2-one, 2-(4-diethylamino-2-hydroxybenzoyl)benzoic acid, p-alkoxycinnamic acid, 2-cyano-3,3-diphenylacrylic acid as well as mixtures thereof and wherein x is an integer selected in the range from 3-16, y is an integer selected in the range from 1-6, and z is an integer selected in the range from 0-10.

The term “topical” is understood here to mean external application to keratinous substances, which are in particular the skin, scalp, eyelashes, eyebrows, nails, mucous membranes and hair.

As the compositions according to the invention are intended for topical application, they comprise a physiologically acceptable medium, that is to say a medium compatible with keratinous substances, such as the skin, mucous membranes, and keratinous fibres. In particular the physiologically acceptable medium is a cosmetically acceptable carrier.

The term cosmetically acceptable carrier refers to all carriers and/or excipients and/or diluents conventionally used in cosmetic compositions.

Preferred topical compositions according to the invention are skin care preparations, decorative preparations, and functional preparations.

Examples of skin care preparations are, in particular, light protective preparations, anti-ageing preparations, preparations for the treatment of photo-ageing, body oils, body lotions, body gels, treatment creams, skin protection ointments, skin powders, moisturizing gels, moisturizing sprays, face and/or body moisturizers, skin-tanning preparations (i.e. compositions for the artificial/sunless tanning and/or browning of human skin), for example self-tanning creams as well as skin lightening preparations.

Examples of decorative preparations are, in particular, lipsticks, eye shadows, mascaras, dry and moist make-up formulations, rouges and/or powders.

Examples of functional preparations are cosmetic or pharmaceutical compositions containing active ingredients such as hormone preparations, vitamin preparations, vegetable extract preparations, anti-ageing preparations, and/or antimicrobial (antibacterial or antifungal) preparations without being limited thereto.

In a particular embodiment the topical compositions according to the invention are light-protective preparations (sun care products), such as sun protection milks, sun protection lotions, sun protection creams, sun protection oils, sun blocks or tropical's or day care creams with a SPF (sun protection factor). Of particular interest are sun protection creams, sun protection lotions, sun protection milks, sun spray preparations and sun protection preparations.

The topical compositions according to the present invention may be in the form of a suspension or dispersion in solvents or fatty substances, or alternatively in the form of an emulsion or micro emulsion (in particular of oil-in-water (O/W-) or water-in-oil (W/O-)type, silicone-in-water (Si/W-) or water-in-silicone (W/Si—)type, PIT-emulsion, multiple emulsion (e.g. oil-in-water-in oil (O/W/O-) or water-in-oil-in-water (W/O/W-)type), pickering emulsion, hydrogel, alcoholic gel, lipogel, one- or multiphase solution or vesicular dispersion or other usual forms, which can also be applied by pens, as masks or as sprays.

The topical compositions according to the present invention are advantageously in the form of an oil-in-water (O/W) emulsion comprising an oily phase dispersed in an aqueous phase in the presence of an O/W emulsifier. The preparation of such O/W emulsions is well known to a person skilled in the art and illustrated in the examples.

If the topical composition according to the invention is an O/W emulsion, then it contains advantageously at least one O/W- or Si/W-emulsifier selected from the list of PEG-30 Dipolyhydroxystearate, PEG-4 Dilaurate, PEG-8 Dioleate, PEG-40 Sorbitan Peroleate, PEG-7 Glyceryl Cocoate, PEG-20 Almond Glycerides, PEG-25 Hydrogenated Castor Oil, Glyceryl Stearate (and) PEG-100 Stearate, PEG-7 Olivate, PEG-8 Oleate, PEG-8 Laurate, PEG-60 Almond Glycerides, PEG-20 Methyl Glucose Sesquistearate, PEG-40 Stearate, PEG-100 Stearate, PEG-80 Sorbitan Laurate, Steareth-2, Steareth-12, Oleth-2, Ceteth-2, Laureth-4, Oleth-10, Oleth-10/Polyoxyl 10 Oleyl Ether, Ceteth-10, Isosteareth-20, Ceteareth-20, Oleth-20, Steareth-20, Steareth-21, Ceteth-20, Isoceteth-20, Laureth-23, Steareth-100, glycerylstearatcitrate, glycerylstearate (self emulsifying), stearic acid, salts of stearic acid, polyglyceryl-3-methylglycosedistearate. Further suitable emulsifiers are phosphate esters and the salts thereof such as cetyl phosphate (Amphisol® A), diethanolamine cetyl phosphate (Amphisol®DEA), potassium cetyl phosphate (Amphisol® K), sodiumcetearylsulfat, sodium glyceryl oleate phosphate, hydrogenated vegetable glycerides phosphate and mixtures thereof. Further suitable emulsifiers are sorbitan oleate, sorbitan sesquioleate, sorbitan isostearate, sorbitan trioleate, Lauryl Glucoside, Decyl Glucoside, Sodium Stearoyl Glutamate, Sucrose Polystearate and Hydrated Polyisobuten. Furthermore, one or more synthetic polymers may be used as an emulsifier. For example, PVP eicosene copolymer, acrylates/C₁₀₋₃₀ alkyl acrylate crosspolymer, acrylates/steareth-20 methacrylate copolymer, PEG-22/dodecyl glycol copolymer, PEG-45/dodecyl glycol copolymer, and mixtures thereof.

The at least one O/W respectively Si/W emulsifier is preferably used in an amount of 0.5 to 10 wt.-%, in particular in the range of 0.5 to 6 wt.-% such as more in particular in the range of 0.5 to 5 wt.-% such as most in particular in the range of 1 to 4 wt.-%, based on the total weight of the composition.

Particular suitable O/W emulsifiers according to the present invention encompass phosphate esters emulsifier of formula (II)

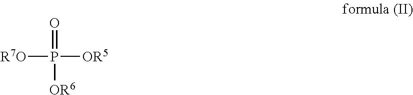

wherein R⁵, R⁶ and R⁷ may be hydrogen, an alkyl of from 1 to 22 carbons, preferably from 12 to 18 carbons; or an alkoxylated alkyl having 1 to 22 carbons, preferably from 12 to 18 carbons, and having 1 or more, preferably from 2 to 25, most preferably 2 to 12, moles ethylene oxide, with the provision that at least one of R⁵, R⁶ and R⁷ is an alkyl or alkoxylated alkyl as previously defined but having at least 6 alkyl carbons in said alkyl or alkoxylated alkyl group.

Monoesters in which R⁵ and R⁶ are hydrogen and R⁷ is selected from alkyl groups of 10 to 18 carbons and alkoxylated fatty alcohols of 10 to 18 carbons and 2 to 12 moles ethylene oxide are preferred. Among the preferred phosphate ester emulsifier are C₈₋₁₀ Alkyl Ethyl Phosphate, C₉₋₁₅ Alkyl Phosphate, Ceteareth-2 Phosphate, Ceteareth-5 Phosphate, Ceteth-8 Phosphate, Ceteth-10 Phosphate, Cetyl Phosphate, C6-10 Pareth-4 Phosphate, C₁₂₋₁₅ Pareth-2 Phosphate, C₁₂₋₁₅ Pareth-3 Phosphate, DEA-Ceteareth-2 Phosphate, DEA-Cetyl Phosphate, DEA-Oleth-3 Phosphate, Potassium cetyl phosphate, Deceth-4 Phosphate, Deceth-6 Phosphate and Trilaureth-4 Phosphate. A particular advantageous phosphate ester emulsifier according to the invention is potassium cetyl phosphate e.g. commercially available as Amphisol® K at DSM Nutritional Products Ltd Kaiseraugst as the overall staining tendency of the benzotriazol derivative is decreased.

Further suitable O/W emulsifiers are polyethyleneglycol (PEG) esters or diesters such as e.g. [INCI Names] PEG-100 Stearate, PEG-30 Dipolyhydroxystearate, PEG-4 Dilaurate, PEG-8 Dioleate, PEG-40 Sorbitan Peroleate, PEG-7 Glyceryl Cocoate, PEG-20 Almond Glycerides, PEG-25 Hydrogenated Castor Oil, PEG-7 Olivate, PEG-8 Oleate, PEG-8 Laurate, PEG-60 Almond Glycerides, PEG-20 Methyl Glucose Sesquistearate, PEG-40 Stearate, PEG-100 Stearate, PEG-80 Sorbitan Laurate.

Particularly preferred according to the present invention is PEG-100 Stearate sold under the tradename Arlacel™ 165 (INCI Glyceryl Stearate (and) PEG-100 Stearate) by Croda.

Another particular suitable class of O/W emulsifiers are non ionic self-emulsifying system derived from olive oil e.g. known as (INCI Name) cetearyl olivate and sorbitan olivate (Chemical Composition: sorbitan ester and cetearyl ester of olive oil fatty acids) sold under the tradename OLIVEM 1000 as the washability of the stains caused by the benzotriazol derivative is improved.

Another class of particularly suitable emulsifiers are polyglycerol esters or diesters of fatty acids also called polyglyceryl ester/diester (i.e. a polymer in which fatty acid(s) is/are bound by esterification with polyglycerine), such as e.g. commercially available at Evonik as Isolan GPS [INCI Name Polyglyceryl-4 Diisostearate/Polyhydroxystearate/Sebacate (i.e. diester of a mixture of isostearic, polyhydroxystearic and sebacic acids with Polyglycerin-4)] or Dehymuls PGPH available at Cognis (INCI Polyglyceryl-2 Dipolyhydroxystearate)

In particular embodiment, the invention relates to topical compositions in the form of O/W emulsions comprising an oily phase dispersed in an aqueous phase in the presence of an O/W emulsifier wherein the benzotriazol derivative of formula (I) is benzotriazolyl dodecyl p-cresol, the Vitamin E or derivative thereof is tocopheryl acetate and the O/W emulsifier is selected from the group consisting of potassium cetyl phosphate, glyceryl stearate (and) PEG-100 Stearate, cetearyl olivate and sorbitan olivate as well as mixtures thereof.

In another particular embodiment, the invention relates to topical compositions in the form of W/O emulsions comprising water dispersed in an oily phase in the presence of an W/O emulsifier wherein the benzotriazol derivative of formula (I) is benzotriazolyl dodecyl p-cresol, the Vitamin E or derivative thereof is tocopheryl acetate and the W/O emulsifier is Polyglyceryl-2 Dipolyhydroxystearate.

In a further embodiment, the invention relates to topical compositions in the form of O/W emulsions comprising an oily phase dispersed in an aqueous phase in the presence of an O/W emulsifier wherein the benzotriazol derivative of formula (I) is a compound of formula (I) wherein R¹ and R⁴ are hydrogen, R² is methyl and R³ is 2,5,5-trimethylhexyloxy, 3,5,5-trimethylhexyloxy, isoamyloxy, 2-ethylhexyloxy or 3,3,5-trimethyl-cyclohexyloxy, the Vitamin E or derivative thereof is tocopheryl acetate and the O/W emulsifier is selected from the group consisting of potassium cetyl phosphate, glyceryl stearate (and) PEG-100 Stearate, cetearyl olivate and sorbitan olivate as well as mixtures thereof. Particularly preferred is a compound of formula (I), wherein R¹ and R⁴ are hydrogen, R² is methyl and R³ is 2-ethylhexyloxy (i.e. 2-(2H-Benzotriazol-2-yl)-6-(2-ethylhexyloxymethyl)-4-methyl-phenol)

In another particular embodiment, the invention relates to topical compositions in the form of W/O emulsions comprising water dispersed in an oily phase in the presence of an W/O emulsifier wherein the benzotriazol derivative of formula (I) is a compound of formula (I) wherein R¹ and R⁴ are hydrogen, R² is methyl and R³ is 2,5,5-trimethylhexyloxy, 3,5,5-trimethylhexyloxy, isoamyloxy, 2-ethylhexyloxy or 3,3,5-trimethyl-cyclohexyloxy, the Vitamin E or derivative thereof is tocopheryl acetate and the W/O emulsifier is Polyglyceryl-2 Dipolyhydroxystearate. Particularly preferred is a compound of formula (I), wherein R¹ and R⁴ are hydrogen, R² is methyl and R³ is 2-ethylhexyloxy (i.e. 2-(2H-Benzotriazol-2-yl)-6-(2-ethylhexyloxymethyl)-4-methyl-phenol)

The topical compositions according to the present invention furthermore advantageously contain at least one co-surfactant such as e.g. selected from the group of mono- and diglycerides and/or fatty alcohols. The co-surfactant is generally used in an amount selected in the range of 0.1 to 10 wt.-%, such as in particular in the range of 0.5 to 5 wt.-%, such as most in particular in the range of 1 to 3 wt.-%, based on the total weight of the composition. Particular suitable co-surfactants are selected from the list of alkyl alcohols such as cetyl alcohol (Lorol C16, Lanette 16) cetearyl alcohol (Lanette O), stearyl alcohol (Lanette 18), behenyl alcohol (Lanette 22), glyceryl stearate, glyceryl myristate (Estol 3650), hydrogenated coco-glycerides (Lipocire Na10) as well as mixtures thereof.

The compositions in form of O/W emulsions according to the invention can be provided, for example, in all the formulation forms for O/W emulsions, for example in the form of serum, milk or cream, and they are prepared according to the usual methods. The compositions which are subject-matters of the invention are intended for topical application and can in particular constitute a dermatological or cosmetic composition, for example intended for protecting human skin against the adverse effects of UV radiation (antiwrinkle, anti-ageing, moisturizing, anti-sun protection and the like).

According to an advantageous embodiment of the invention the compositions constitute cosmetic composition and are intended for topical application to the skin.

Another subject matter of the invention is directed to a method for reducing the sand adherence on skin after application of a topical composition, said method comprising the step of incorporating into said composition vitamin E or a derivative thereof and at least one benzotriazol derivative of formula (I), with all definitions and preferences as given above.

The invention also relates to the use of a benzotriazol derivative of formula (I) with all the definitions and preferences as given above as sand repellent, particularly in combination with vitamin E or a derivative thereof such as particularly tocopheryl acetate.

Finally, a subject-matter of the invention is a method for the cosmetic treatment of keratinous substances such as in particular the skin, characterized in that a composition as defined above is applied to the said keratinous substances such as in particular to the skin. The method is in particular suitable to protect the skin against the adverse effects of UV-radiation such as in particular sun-burn and/or photoageing.

In accordance with the present invention, the compositions according to the invention may comprise further ingredients such as ingredients for skin lightening; tanning prevention; treatment of hyperpigmentation; preventing or reducing acne, wrinkles, lines, atrophy and/or inflammation; chelators and/or sequestrants; anti-cellulites and slimming (e.g. phytanic acid), firming, moisturizing and energizing, self tanning, soothing, as well as agents to improve elasticity and skin barrier and/or further UV-filter substances and carriers and/or excipients or diluents conventionally used in topical compositions. If nothing else is stated, the excipients, additives, diluents, etc. mentioned in the following are suitable for topical compositions according to the present invention. The necessary amounts of the cosmetic and dermatological adjuvants and additives can, based on the desired product, easily be determined by the skilled person. The additional ingredients can either be added to the oily phase, the aqueous phase or separately as deemed appropriate. The mode of addition can easily be adapted by a person skilled in the art.

The cosmetically active ingredients useful herein can in some instances provide more than one benefit or operate via more than one mode of action.

The topical cosmetic compositions of the invention can also contain usual cosmetic adjuvants and additives, such as preservatives/antioxidants, fatty substances/oils, water, organic solvents, silicones, thickeners, softeners, emulsifiers, sunscreens, antifoaming agents, moisturizers, aesthetic components such as fragrances, surfactants, fillers, sequestering agents, anionic, cationic, nonionic or amphoteric polymers or mixtures thereof, propellants, acidifying or basifying agents, dyes, colorings/colorants, abrasives, absorbents, essential oils, skin sensates, astringents, antifoaming agents, pigments or nanopigments, e.g. those suited for providing a photoprotective effect by physically blocking out ultraviolet radiation, or any other ingredients usually formulated into cosmetic compositions. Such cosmetic ingredients commonly used in the skin care industry, which are suitable for use in the compositions of the present invention are e.g. described in the CTFA Cosmetic Ingredient Handbook, Second Edition (1992), The Cosmetic, Toiletry and Fragrance Association, Inc. without being limited thereto.

The necessary amounts of the cosmetic and dermatological adjuvants and additives can—based on the desired product—easily be chosen by a skilled person in this field and will be illustrated in the examples, without being limited hereto.

Of course, one skilled in this art will take care to select the above mentioned optional additional compound or compounds and/or their amounts such that the advantageous properties intrinsically associated with the combination in accordance with the invention are not, or not substantially, detrimentally affected by the envisaged addition or additions.

The topical compositions according to the invention in general have a pH in the range of 3 to 10, preferably a pH in the range of 4 to 8 and most preferably a pH in the range of 4 to 7. The pH can easily be adjusted as desired with suitable acids such as e.g. citric acid or bases such as NaOH, Triethanolamine (TEA Care), Tromethamine (Trizma Base) and Aminomethyl Propanol (AMP-Ultra PC 2000) according to standard methods in the art.

The following examples are provided to further illustrate the compositions and effects of the present invention. These examples are illustrative only and are not intended to limit the scope of the invention in any way.

### EXAMPLE 1

### Staining Properties

### Compositions

The compositions as outlined in table 1 were prepared according to standard methods.

TABLE 1
Compositions

| | 1A | | 2A | | 3A | |
|---|---|---|---|---|---|---|
| | (Refer- | | (Refer- | | (Refer- | |
| | ence) | 1B | ence) | 2B | ence) | 3B |
| Trade Name | wt.-% | wt.-% | wt.-% | wt.-% | wt.-% | wt.-% |
| Phenonip | 0.80 | 0.80 | 0.80 | 0.80 | 0.80 | 0.80 |
| Keltrol CG-T | 0.30 | 0.30 | 0.30 | 0.30 | 0.30 | 0.30 |
| Finsolv TN | 30.00 | 30.00 | 30.00 | 30.00 | 30.00 | 30.00 |
| Arlacel 165 | 5.00 | 5.00 | | | | |
| Olivem 1000 | | | 5.00 | 5.00 | | |
| Ampisol K | | | | | 5.00 | 5.00 |
| Lanette O | 1.50 | 1.50 | | | 1.50 | 1.50 |
| Tinogard TL | 8.00 | 8.00 | 8.00 | 8.00 | 8.00 | 8.00 |
| Tocopherol | | 2.00 | | 2.00 | | 2.00 |
| Acetate | | | | | | |
| Water | Ad 100 | Ad 100 | Ad 100 | Ad 100 | Ad 100 | Ad 100 |

| | 4A | | 5A | |
|---|---|---|---|---|
| | (Reference) | 4B | (Reference) | 5B |
| Trade Name | wt.-% | wt.-% | wt.-% | wt.-% |
| Phenonip | | | 0.80 | 0.80 |
| Keltrol CG-T | 0.30 | 0.30 | 0.30 | 0.30 |
| Finsolv TN | 30.00 | 30.00 | 30.00 | 30.00 |
| Dehymuls PGPH | 6.00 | 6.00 | | |
| Olivem 1000 | | | 5.00 | 5.00 |
| Lanette O | | | 1.50 | 1.50 |
| Tinogard TL | 8.00 | 8.00 | | |
| 2-(2H-Benzotriazol- | | | 8.00 | 8.00 |
| 2-yl)-6-(2- | | | | |
| ethylhexyloxymethyl)- | | | | |
| 4-methyl-phenol | | | | |
| (Chemical name)* | | | | |
| Tocopherol Acetate | | 2.00 | | 2.00 |
| Magnesiumsulfate | 0.50 | 0.50 | | |
| heptahydrate | | | | |
| Water | Ad 100 | Ad 100 | Ad 100 | Ad 100 |

*i.e. a compound of formula (I), wherein R¹ and R⁴ are hydrogen, R² is methyl and R³ is 2-ethylhexyloxy

### Wash Trials (Treatment)

On a textile cloth (Jersey by the company Testex “Prüftextilien” Windheckenweg 53, D-53902 Bad Münstereifel) squares of 5 cm×5 cm size were indicated. Afterwards 1 ml of the respective formulation was applied homogenously onto this field.

Afterwards, the cloth was dried for 30 min at 40° C. before the residual emulsion was removed with a paper towel. Then the cloth was washed at 40° C. with a washing powder (Persil Color Megaperls) in a washing machine using a washing program for fine textiles. After taking the cloth out of the washing machine it was air-dried.

### Lab Measurement

The treated cloth is laid onto one layer of the white, untreated textile cloth and the L,a,b value is determined with a Minolta Spectrophotometer CM-3600d.

As reference for the L,a,b measurement an untreated cloth, treated in the same manner as the samples (i.e. washed & dried but without application of a formulation) is used. The AE value is calculated according to the following formula:

ΔE=\sqrt{(L₁−L₂)²+(a₁−a₂)²+(b₁−b₂)}²

| No. | Emulsifier | ΔE |
|---|---|---|
| 1A (Reference sample) | Arlacel 165 | 6.1 |
| 1B | Arlacel 165 | 6.0 |
| 2A (Reference sample) | Olivem 1000 | 6.2 |
| 2B | Olivem 1000 | 4.5 |
| 3A (Reference sample) | Amphisol K | 5.1 |
| 3B | Amphisol K | 5.0 |
| 4A (Reference sample) | Dehymuls PGPH | 1.43 |
| 4B | Dehymuls PGPH | 1.09 |
| 5A (Reference sample) | Olivem 1000 | 5.25 |
| 5B | Olivem 1000 | 4.82 |

As can be retrieved from the table the addition of tocopheryl acetate results in an overall decrease of the staining tendency reflected by the reduced ΔE value.

Furthermore, it can be retrieved that the use of an alky phosphate emulsifier such as Amphisol K as well as a polyglycerol ester/diester of fatty acid emulsifier such as Dehymuls PGPH results in an overall reduced staining tendency of the benzotriazol derivative. The use of non ionic self-emulsifying system derived from olive oil such as Olivem 1000 (INCI cetearyl olivate and sorbitan olivate) as well as the polyglycerol ester/diester of fatty acid emulsifier such as Dehymuls PGPH furthermore result in an improved washability of the stains.

### EXAMPLE 2

### Sand Repellent Properties

50 mg of the respective formulation as outlined in table 2 were applied on PMMA Schönberg plates (5×5 cm). Plates are dried at 43° C. for 15 minutes. 2.5 g sand were distributed on the plates. For removal of excess sand the plates were tilt (about 45°, left and right side). The sticky sand residue on each plate was determined through weighting. The results are presented in table 2.

The reduction in the sand adherence was determined as:

Δ Adherence[%]={(Adherence Sample[mg]−Adherence Reference[mg])/Adherence Sample[mg]}*100%.

TABLE 2
Formulation Set 1 with 8% Tinogard TL & 2% Tocopherol Acetate

| | 5A | | 6A | | 7A | |
|---|---|---|---|---|---|---|
| | (Refer- | | (Refer- | | (Refer- | |
| | ence) | 5B | ence) | 6B | ence) | 7B |
| Trade Name | wt.-% | | wt.-% | | wt.-% | |
| Phenonip | 0.80 | 0.80 | 0.80 | 0.80 | 0.80 | 0.80 |
| Keltrol CG-T | 0.30 | 0.30 | 0.30 | 0.30 | 0.30 | 0.30 |
| Finsolv TN | 30.00 | 30.00 | 30.00 | 30.00 | 30.00 | 30.00 |
| Arlacel 165 | 5.00 | 5.00 | | | | |
| Olivem 1000 | | | 5.00 | 5.00 | | |
| Ampisol K | | | | | 5.00 | 5.00 |
| Lanette O | 1.50 | 1.50 | | | 1.50 | 1.50 |
| Tinogard TL | | 8.00 | | 8.00 | | 8.00 |
| Tocopherol Acetate | | 2.00 | | 2.00 | | 2.00 |
| Water | Ad 100 | | Ad 100 | | Ad 100 | |
| Sand adherence [mg] | 1.41 | 1.30 | 1.50 | 1.19 | 1.32 | 1.29 |
| Δ Adherence [%] | −8% | | −21% | | −2% | |
| | 8A | | 9A | | 10A | |
| | (Refer- | | (Refer- | | (Refer- | |
| | ence) | 8B | ence) | 9B | ence) | 10B |
| Trade Name | wt.-% | | wt.-% | | wt.-% | |
| Phenonip | | | 0.80 | 0.80 | 0.80 | 0.80 |
| Keltrol CG-T | 0.30 | 0.30 | 0.30 | 0.30 | 0.30 | 0.30 |
| Finsolv TN | 30.00 | 30.00 | 30.00 | 30.00 | 30.00 | 30.00 |
| Dehymuls PGPH | 6.00 | 6.00 | | | | |
| Brij 72 | | | 2.5 | 2.5 | | |
| Brij 721 | | | 2.5 | 2.5 | | |
| Pemulen TR2 | | | | | 0.40 | 0.40 |
| Lanette O | | | 1.00 | 1.00 | | |
| Tinogard TL | | 8.00 | | 8.00 | | 8.00 |
| Tocopherol Acetate | | 2.00 | | 2.00 | | 2.00 |
| TEA Care | | | | | 0.40 | 0.40 |
| Magnesiumsulfate | 0.50 | 0.50 | | | | |
| heptahydrate | | | | | | |
| Water | Ad 100 | | Ad 100 | | Ad 100 | |
| Sand adherence [mg] | 1.51 | 1.41 | 1.55 | 1.41 | 1.59 | 1.38 |

| | | | |
|---|---|---|---|
| Δ Adherence [%] | −7% | −9% | −13% |

TABLE 3
Formulation Set 2 with 4% Tinogard TL,
2% Tocopherol Acetate & 4% PARSOL 1789

| | 11A | | 12A | | 13A | |
|---|---|---|---|---|---|---|
| | (Refer- | | (Refer- | | (Refer- | |
| | ence) | 11B | ence) | 12B | ence) | 13B |
| Trade Name | wt.-% | | | | | |
| Phenonip | 0.80 | 0.80 | 0.80 | 0.80 | 0.80 | 0.80 |
| Keltrol CG-T | 0.30 | 0.30 | 0.30 | 0.30 | 0.30 | 0.30 |
| Finsolv TN | 30.00 | 30.00 | 30.00 | 30.00 | 30.00 | 30.00 |
| PARSOL 1789 | 4.00 | 4.00 | 4.00 | 4.00 | 4.00 | 4.00 |
| Arlacel 165 | 5.00 | 5.00 | | | | |
| Olivem 1000 | | | 5.00 | 5.00 | | |
| Ampisol K | | | | | 5.00 | 5.00 |
| Lanette O | 1.50 | 1.50 | | | 1.50 | 1.50 |
| Tinogard TL | | 4.00 | | 4.00 | | 4.00 |
| Tocopherol Acetate | | 2.00 | | 2.00 | | 2.00 |
| Water | Ad 100 | | | | | |
| Sand adherence [mg] | 1.54 | 1.48 | 1.43 | 1.33 | 1.17 | 0.94 |

| | | | |
|---|---|---|---|
| Δ Adherence [%] | −23% | −7% | −19% |

TABLE 4
Formulation Set 3 with 8% 2-(2H-Benzo-triazol-2-yl)-6-(2-ethylhexyl-
oxy-methyl)-4-methyl-phenol & 2% Tocopherol Acetate

| | |
|---|---|
| 14A (Reference) | 14B |

| | |
|---|---|
| Trade Name | wt.-% |

| | | |
|---|---|---|
| Phenonip | 0.80 | 0.80 |
| Keltrol CG-T | 0.30 | 0.30 |
| Finsolv TN | 30.00 | 30.00 |
| Ampisol K | 5.00 | 5.00 |
| Lanette O | 1.50 | 1.50 |
| 2-(2H-Benzo-triazol-2-yl)-6-(2-ethyl- | | 8 |
| hexyl-oxy-methyl)-4-methyl-phenol* | | |
| Tocopherol Acetate | | 2.00 |

| | |
|---|---|
| Water | Ad 100 |

| | | |
|---|---|---|
| Sand adherence [mg] | 1.32 | 1.15 |

| | |
|---|---|
| Δ Adherence [%] | −13% |

*benzotriazol derivative of formula (I): 2-(2H-Benzotriazol-2-yl)-6-(2-ethylhexyloxymethyl)-4-methyl-phenol (i.e. a compound of formula (I), wherein R¹ and R⁴ are hydrogen, R² is methyl and R³ is 2-ethylhexyloxy)

TABLE 5
Formulation Set 4 with 4 2-(2H-Benzo-triazol-
2-yl)-6-(2-ethylhexyl-oxy-methyl)-4-methyl-phenol*,
2% Tocopherol Acetate & 4% PARSOL 1789

| | |
|---|---|
| 15A (Reference) | 15B |

| | |
|---|---|
| Trade Name | wt.-% |

| | | |
|---|---|---|
| Phenonip | 0.80 | 0.80 |
| Keltrol CG-T | 0.30 | 0.30 |
| Finsolv TN | 30.00 | 30.00 |
| PARSOL 1789 | 4.00 | 4.00 |
| Ampisol K | 5.00 | 5.00 |
| Lanette O | 1.50 | 1.50 |
| 2-(2H-Benzo-triazol-2-yl)-6-(2-ethyl- | | 4.00 |
| hexyl-oxy-methyl)-4-methyl-phenol* | | |
| Tocopherol Acetate | | 2.00 |

| | |
|---|---|
| Water | Ad 100 |

| | | |
|---|---|---|
| Sand adherence [mg] | 1.17 | 1.10 |

| | |
|---|---|
| Δ Adherence [%] | −6% |

*benzotriazol derivative of formula (I): 2-(2H-Benzotriazol-2-yl)-6-(2-ethylhexyloxymethyl)-4-methyl-phenol (i.e. a compound of formula (I), wherein R¹ and R⁴ are hydrogen, R² is methyl and R³ is 2-ethylhexyloxy)

As can be retrieved from the results in table 2, 3, 4 and 5 the compositions according to the present invention also exhibit a reduced sand-adherence.

1. A topical composition comprising vitamin E or a derivative thereof and at least one benzotriazol derivative of formula (I) 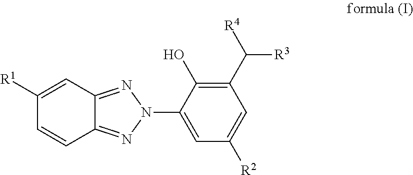
wherein
R¹ is hydrogen; C₁₋₅alkyl; C₁₋₅alkoxy or halogen; preferably hydrogen or chloride, most preferably hydrogen;
R² is hydrogen; C₁₋₂₀alkyl; C₁₋₅alkoxy; C₁₋₅alkoxycarbonyl; C₅₋₁₀cycloalkyl; C₆₋₁₀aryl or aralkyl; preferably hydrogen or C₁₋₅alkyl, most preferably methyl;
R³ is C₁₋₂₀alkyl, C₅₋₁₀cycloalkyl, C₁₋₂₀alkoxy or C₅₋₁₀cycloalkoxy, preferably C₅₋₁₅alkyl or C₅₋₁₅alkoxy; and
R⁴ is hydrogen or C₁₋₅alkyl, preferably hydrogen
characterized in that the benzotriazol derivative of formula (I) is present in an amount ranging from 1 to 20 wt.-%, based on the total weight of the composition.

2. The topical composition according to claim 1, characterized in that the vitamin E or a derivative thereof is used in an amount selected in the range of 0.05 to 25 wt.-%, based on the total weight of the composition.

3. The topical composition according to claim 1,
characterized in that the benzotriazol derivative is used in an amount selected in the range of 2 to 20 wt.-%, based on the total weight of the composition.

4. The topical composition according to claim 1, characterized in that the benzotriazol compound of formula (I) is a compound wherein R¹ and R⁴ are hydrogen, R² is methyl and R³ is 2,5,5-trimethylhexyloxy, 3,5,5-trimethylhexyloxy, isoamyloxy, 2-ethylhexyloxy, 3,3,5-trimethyl-cyclohexyloxy or undecyl.

5. The topical composition according to claim 1, characterized in that the Vitamin E or derivative thereof is tocopheryl acetate.

6. The topical composition according to claim 1, characterized in that the topical composition is an O/W emulsion comprising an oily phase dispersed in an aqueous phase in the presence of an O/W emulsifier.

7. The topical composition according to claim 6, characterized in that the O/W emulsifier is selected from the group consisting of phosphate esters, polyethyleneglycol esters or diesters, stearates and/or polymeric O/W emulsifier.

8. The topical composition according to claim 7, characterized in that the O/W emulsifier is selected from the group consisting of potassium cetyl phosphate, glyceryl stearate, PEG-100 stearate, cetearyl olivate and/or sorbitan olivate.

9. The topical composition according to claim 6, characterized in that the amount of O/W emulsifier is selected in the range of 0.5 to 10 wt.-%, based on the total weight of the composition.

10. The topical composition according to claim 1, characterized in that the composition comprises at least one co-surfactant in an amount selected in the range of 0.1 to 10 wt.-%, based on the total weight of the composition.

11. The topical composition according to claim 10, characterized in that the co-surfactant is selected from the group consisting of cetyl alcohol, cetearyl alcohol, stearyl alcohol, behenyl alcohol, glyceryl stearate, glyceryl myristate, hydrogenated coco-glycerides and mixtures thereof.

12. The topical composition according to claim 1, characterized in that the topical composition is a W/O emulsion comprising a water phase dispersed in an oily phase in the presence of a W/O emulsifier.

13. The topical composition according to claim 12, characterized in that the W/O emulsifier is Polyglyceryl-2 Dipolyhydroxystearate.

14. Use of Vitamin E or a derivative thereof for reducing cloth staining caused by a benzotriazol derivative of formula (I) 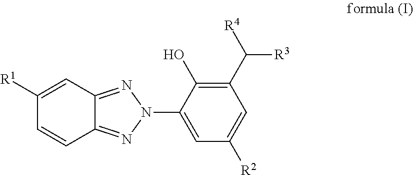
wherein
R¹ is hydrogen; C₁₋₅alkyl; C₁₋₅alkoxy or halogen; preferably hydrogen or chloride; most preferably hydrogen;
R² is hydrogen; C₁₋₂₀alkyl; C₁₋₅alkoxy; C₁₋₅alkoxycarbonyl; C₅₋₁₀cycloalkyl; C₆₋₁₀aryl or aralkyl; preferably hydrogen or C₁₋₅alkyl; most preferably methyl;
R³ is C₁₋₂₀alkyl; C₅₋₁₀cycloalkyl; C₁₋₂₀alkoxy or C₅₋₁₀cycloalkoxy, preferably C₅₋₁₅alkyl or C₅₋₁₅alkoxy; and
R⁴ is hydrogen or C₁₋₅alkyl; preferably hydrogen

15. Method for reducing stains on clothes caused by the use of topical compositions comprising a benzotriazol derivative of formula (I) 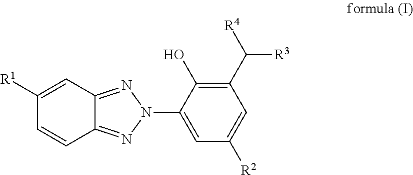
wherein
R¹ is hydrogen; C₁₋₅alkyl; C₁₋₅alkoxy or halogen; preferably hydrogen or chloride; most preferably hydrogen;
R² is hydrogen; C₁₋₂₀alkyl; C₁₋₅alkoxy; C₁₋₅alkoxycarbonyl; C₅₋₁₀cycloalkyl; C₆₋₁₀aryl or aralkyl; preferably hydrogen or C₁₋₅alkyl; most preferably methyl;
R³ is C₁₋₂₀alkyl, C₅₋₁₀cycloalkyl; C₁₋₂₀alkoxy or C₅₋₁₀cycloalkoxy; preferably C₅₋₁₅alkyl or C₅₋₁₅alkoxy; and
R⁴ is hydrogen or C₁₋₅alkyl; preferably hydrogen
said method comprising the addition of Vitamin E or a derivative thereof into said topical composition.

16. A method for the cosmetic treatment of keratinous substances such as in particular the skin, characterized in that a composition as defined in claim 1 is applied to the said keratinous substances such as in particular to the skin.

17. Use of a benzotriazol derivative of formula (I) 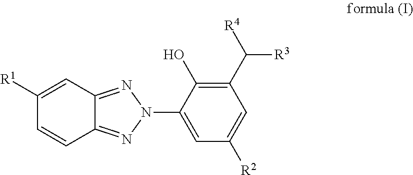
wherein
R¹ is hydrogen; C₁₋₅alkyl; C₁₋₅alkoxy or halogen; preferably hydrogen or chloride, most preferably hydrogen;
R² is hydrogen; C₁₋₂₀alkyl; C₁₋₅alkoxy; C₁₋₅alkoxycarbonyl; C₅₋₁₀cycloalkyl; C₆₋₁₀aryl or aralkyl; preferably hydrogen or C₁₋₅alkyl, most preferably methyl;
R³ is C₁₋₂₀alkyl, C₅₋₁₀cycloalkyl, C₁₋₂₀alkoxy or C₅₋₁₀cycloalkoxy, preferably C₅₋₁₅alkyl or C₅₋₁₅alkoxy; and
R⁴ is hydrogen or C₁₋₅alkyl, preferably hydrogen as sand repellent.
